(19) United States
(12) Patent Application Publication
(10) Pub. No.: US 2015/0080942 A1
(43) Pub. Date: Mar. 19, 2015

(54) System and Method for Assisted Manual Compression of Blood Vessel

(71) Applicant: Silk Road Medical, Inc., Sunnyvale, CA (US)

(72) Inventors: Michi E. Garrison, Sunnyvale, CA (US); Tony M. Chou, Sunnyvale, CA (US)

(21) Appl. No.: 14/476,651

(22) Filed: Sep. 3, 2014

Related U.S. Application Data
(60) Provisional application No. 61/877,860, filed on Sep. 13, 2013.

(51) Int. Cl. A61B 17/132; A61B 8/06; A61B 17/135

(52) U.S. Cl. CPC A61B 17/1325; A61B 17/135; A61B 17/1355; A61B 8/06
USPC 606/202; 606/203

(57) ABSTRACT

A device for applying pressure to a region of a person's neck includes a belt having a length sized to fit around the neck and a compression element coupled to the belt. The compression element is sized and shaped to exert a compressive force onto an area of the neck when the belt is tensioned.

### REFERENCE TO PRIORITY DOCUMENT

This application claims the benefit of priority of co-pending U.S. Provisional Patent Application Ser. No. 61/877,860, entitled “System and Method for Assisted Manual Compression of Blood Vessel” filed Sep. 13, 2013. The filing dates and subject matter of the above-noted application is incorporated by reference in its entirety by reference thereto.

### BACKGROUND

Direct access of the common carotid artery has been identified as a favorable access site for carotid, intracranial and cerebral vascular interventions. Direct access of the carotid artery avoids the peripheral and aortic anatomy required in the more common femoral arterial access site to these same anatomic targets, thereby reducing a major source of access-related procedural time and complications. Achieving hemostatic closure at the carotid artery access site has some differences relative to the femoral site.

FIG. 1 shows a cross-sectional view of the neck in the region of the carotid artery. The carotid artery is closer to the skin as compared to the femoral artery. Thus, it is easier to achieve closure in the carotid artery through manual compression the majority of the time. In many cases, simple manual compression of the access site may be an acceptable alternative to using a vessel closure device. However, because of the proximity of the carotid artery to the airway passage, a major hematoma at this site may partially or fully block off the air passage and may be potentially life threatening. It is important that flow is maintained through the carotid artery during and after closure, especially in stroke patients who reply on optimal flow to the brain to achieve the best clinical outcomes.

However, manual compression can take time, often up to 30-60 minutes. Patients may have IV thrombolytic drugs and heparin, which may interfere with blood coagulation, and this may take even longer to achieve hemostasis.

Devices for achieving hemostasis may be used to reduce the time to achieve hemostasis and/or to increase the rate of successful hemostasis at the access site. Hemostasis devices generally fall into three categories: (1) vascular access site closure devices; (2) external compression devices; and (3) topical hemostasis pads or patches. Because the femoral artery is by far the most common access site for cardiovascular and neurovascular interventions, most of these devices have been designed for use with the femoral artery rather than the with the carotid artery. Most vessel closure devices available today are not suitable for the carotid arterial access site. For example, the PERCLOSE PROGLIDE suture closure device (Abbott Vascular) is designed to enter the arteriotomy at the common femoral artery level. The device has an extended portion distal to a closure mechanism element that is advanced through the iliac arteries to the descending aorta. This device with its long distal portion would not work in a carotid artery access site, as the cervical vessels do not have the long segments present in the femoral artery. Other vessel closure devices, such as the ANGIOSEAL device (St. Jude Medical) leave behind a biodegradable element inside the artery. The consequence of embolization of any intravascular element left in the carotid artery is more severe than in the femoral and may lead to major stroke or death. Moreover, closure devices that rely on bioaborbable plugs may mask oozing hematomas, which may lead to compromised airway passages over time.

External compression devices are used either in place of or in conjunction with vessel closure devices. When used in conjunction with vessel closure devices, external compression devices may reduce oozing and/or increase security of hemostasis at the access site. These external compression devices include clamps and inflatable pads, such as the FEMOSTOP (St. Jude). Current external compression devices are optimized for the femoral artery access site and are not suitable for the carotid artery. These devices are designed to blindly apply direct pressure on the femoral artery, are not designed to compress while allowing visualization to confirm flow through the artery, and will not fit around the neck. In addition, there is no feedback if hemostasis is lost or if artery flow is blocked.

In all cases, ultrasound imaging may be utilized to view the artery and verify hemostasis before or after device application. The ultrasound probe is placed on the artery and the puncture site can be visualized, as well as the flow through the artery.

### SUMMARY

Disclosed is an artery compression device that includes a medical tourniquet or belt having a holder configured to hold and position an ultrasound probe over a carotid artery puncture site or access site. In an embodiment, the probe functions as both the compression element and the sensor to monitor blood flow and hemostasis. The belt may be adjustable in size.

In one aspect, there is disclosed a device for applying pressure to a region of a person's neck, comprising: a belt having a length sized to fit around the neck; and a compression element coupled to the belt, the compression element sized and shaped to exert a compressive force onto an area of the neck when the belt is tensioned.

Other features and advantages should be apparent from the following description of various embodiments, which illustrate, by way of example, the principles of the invention.

### BRIEF DESCRIPTION OF THE FIGURES

FIG. 1 shows a cross-sectional view of the neck in the region of the carotid artery.

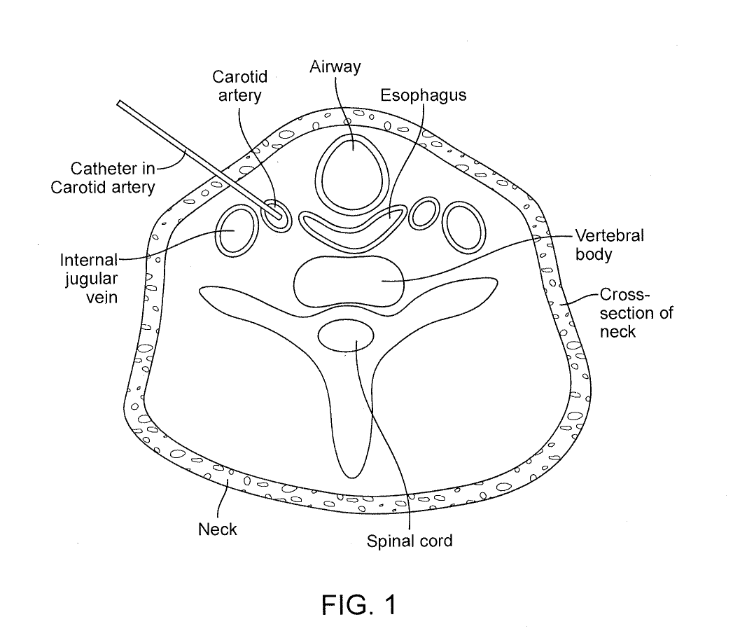

FIG. 2 shows a first embodiment of an artery compression device.

FIG. 3 shows another embodiment of the compression device.

FIG. 4 shows an embodiment of the compression device having a holder structure configured to hold an ultrasound probe compression element.

FIG. 5 shows an embodiment of the compression device with a seat for an ultrasound probe.

FIG. 6 shows an embodiment of the compression device that includes a holder structure having an inflatable compression tip.

### DETAILED DESCRIPTION

In view of the forgoing, there is a need for devices and methods that can achieve hemostasis through “hands free” external compression, or assist in hemostasis with a vessel closure device, and that is suitable for use at a carotid artery access site (i.e., in the region of the neck). In addition, there is a need for simultaneous ultrasound visualization of the access site to verify both hemostasis and flow though the artery. In addition, there is a need to couple the ultrasound information with the adjustable compression such that changes in patient position or device movement can be accommodated with feedback to maintain hemostasis and flow through the artery.

In addition, after hemostasis is achieved, there may be a need to maintain the patient's neck in a mobilized state or semi-mobilized state while allowing visual monitoring of the access site for development of a hematoma. Thus, there is a need for methods and devices that may also restrict neck movement and may also allow direct visual access to the access site.

Disclosed is an artery compression device that includes a medical tourniquet or belt having a holder configured to hold and position an ultrasound probe over a carotid artery puncture site or access site. In an embodiment, the probe functions as both the compression element and the sensor to monitor blood flow and hemostasis. The belt may be adjustable in size.

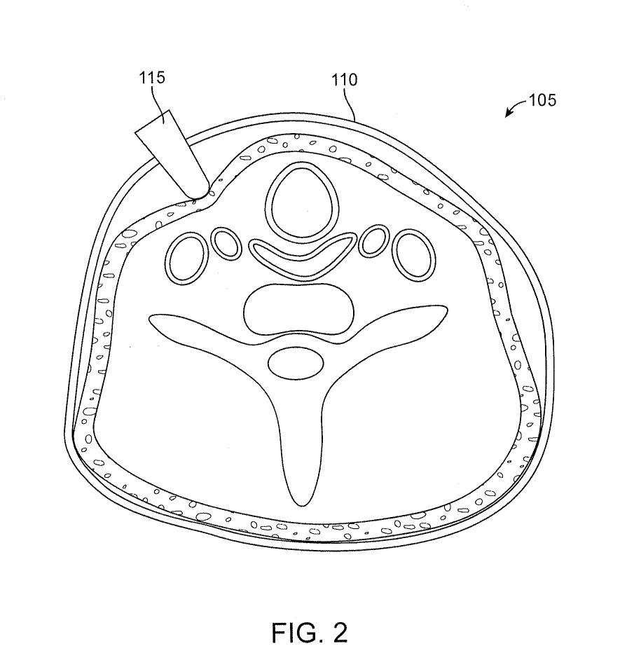

FIG. 2 shows a first embodiment of the compression device 105, which includes a belt 110. The view of FIG. 2 is a cross-sectional view looking toward the long axis of the spine. The belt 110 has a length such that it can surround the patient's neck. The length may be adjusted such that the belt is in a state of tension so that the belt exerts pressure or compression on the neck. The belt 110 is fixedly or removably attached to a localized compression element 115 that serves to provide localized compression or pressure to a region of the neck such as when the belt is tensioned. Thus, the compression element 115 is attached to the belt 110 in a position that exerts pressure or compression on the neck particularly in the area of the carotid artery. The compression element 115 may have a shape that is configured to provide localized pressure to the neck.

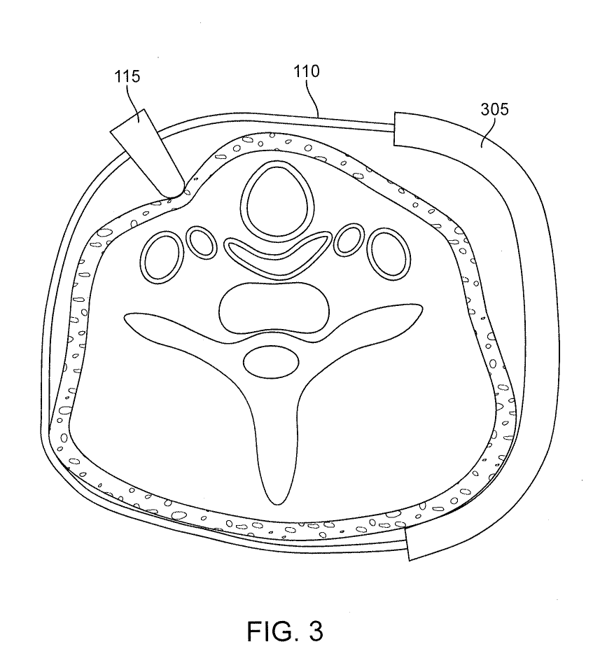

In an embodiment shown in FIG. 3, the belt includes or is coupled to a rigid support structure 305 that is sized and shaped to aid in patient comfort and provide counter traction to the compression site of the compression element. The support structure 305 may be an elongated body that is positioned along the belt 110 on the opposite side of the neck from the compression element, so as to provide a counter support for the direction of force of the compression element. The support structure 305 may have a rounded, padded, or contoured surface that provides or increases comfort to the wearer's neck.

As mentioned, the belt 110 is tensioned such that the compression element 115 exerts pressure to the region of the carotid artery. The support structure 305 is also sized and shaped to protect compression of the belt on the trachea, carotid artery, jugular vein, and/or other structures on the opposite side of the neck when the belt is tensioned. The support structure 305 or other portion of the compression device 105 may include a tension adjustment mechanism or means that permits a user to adjust the belt tension and/or size.

In an embodiment, the compression device 105 includes a component that is configured to measure tension such that the tension may be adjusted over time to one or more pre-determined settings. For example, an initial tension may be on the high end, and the tension may be reduced over time based on a pre-determined protocol or on observation of site hemostasis to lower settings.

In an embodiment, the compression element is an ultrasound probe. In a further embodiment, the ultrasound probe may be attached to a feedback alarm that sounds or is connected to a nurse station alert if the puncture site is not hemostatic or if flow through the carotid artery is stopped.

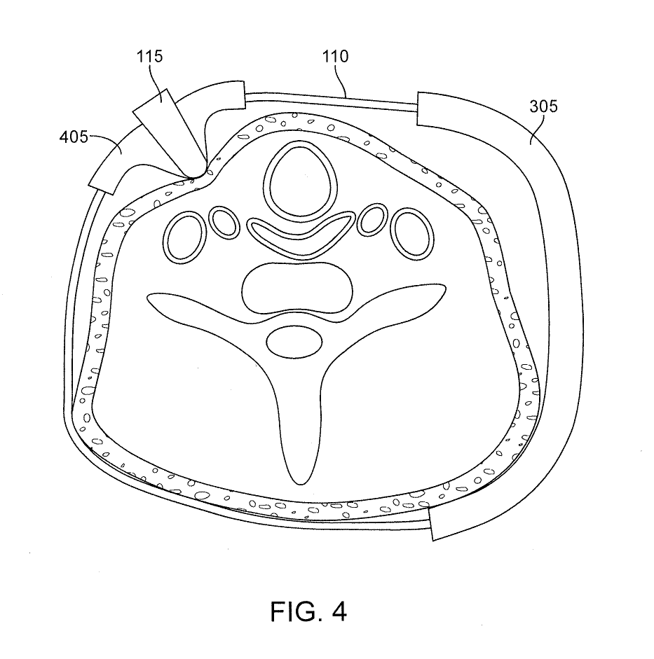

As shown in the embodiment of FIG. 4, the compression device 105 may includes a holder structure 405 that is configured to hold or otherwise secure the ultrasound probe compression element 115 relative to the belt 110 and the neck. The holder structure 405 can hold the ultrasound probe in a predetermined and/or adjustable position relative to the neck. The ultrasound probe 115 may be fixedly or removably mountable in the holder structure 405. In the embodiment of FIG. 4, ultrasound probe 115 extends through the holder structure 405 and actually contacts the neck such that the ultrasound probe itself provides the compressive force.

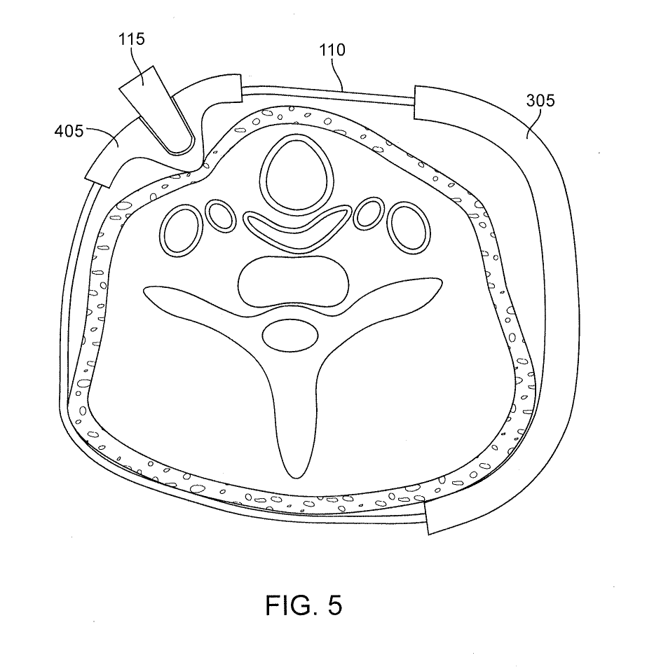

In another embodiment shown in FIG. 5, the holder structure 305 has a seat in which the ultrasound probe 115 is fixedly or removably positioned. The ultrasound probe 115 does not directly contact the neck but rather extends only partway into the holder structure 405. In this embodiment, the holder structure 405 contacts the neck and serves as the compression element that provides the compressive force to the neck.

The embodiment of FIG. 5 may be desirable if the ultrasound probe shape is not the best shape for compression of the artery, or if the position of the probe to monitor flow is different from the optimal position to apply compression. In this configuration, the probe position in the holder structure 405 can be adjustable to obtain the best possible flow signal while compression is being applied. In an embodiment, the holder can adjust the angle of the probe. For example, optimal angle of the probe may be different depending on if the carotid artery is the left carotid or right carotid artery, as well as dependent on individual patient anatomy. In an embodiment, the holder structure 405 is clear and allows visual examination of the access site when the ultrasound probe is removed, without releasing compression.

The ultrasound probe 115 may also be communicatively coupled to the tension adjustment mechanism such that the belt 110 automatically adjusts to maintain hemostasis while flow through carotid artery based on input from the ultrasound probe 115. This may be achieved, for example, by a signal processor that is connected to a servo device in the belt that can lengthen or shorten the length of the belt, or in the holder structure such that the probe can be moved up or down, or other directions as needed to maintain hemostasis or maintain a good flow signal. The signal processor receives data from the ultrasound probe and determines if the belt needs to be lengthened or shortened, or if the probe needs to be moved up or down or otherwise adjusted, and then relays the command to the servo mechanism to make the adjustment.

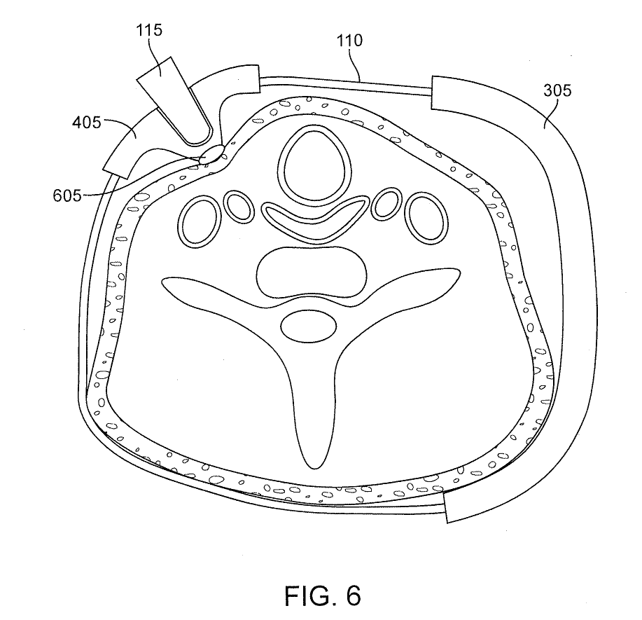

With reference to FIG. 6, another embodiment of the compression device 105 includes a holder structure 405 having an inflatable compression tip 605 that contacts the skin to provide compression. The compression tip 605 has an inflation volume that can be adjusted to get hemostasis while still allowing flow through the carotid artery. In this embodiment, both the belt length and tension, and inflatable tip volume may be adjusted to achieve the desired amount of compression.

The ultrasound probe 115 may also be connected to a device that can apply variable pressure and/or volume to the inflatable compression tip 605, such that the inflation volume automatically adjusts to maintain hemostasis and/or flow through carotid artery. This may be achieved, for example, by coupling the device to a signal processor that is connected to an adjustable fluid source. The adjustable fluid source controls the pressure or volume in the inflatable compression tip 605. The signal processor receives data from the ultrasound probe and determines if the compression needs to be adjusted, and conveys a command to the adjustable fluid source.

In another embodiment, the holder structure 405 may be formed of one or more rigid segments or sections that restrict the movement of the neck. Thus, even after hemostasis is achieved, the wearer of the device has restricted neck movement, thus reducing the chance that the access site will re-open. When the ultrasound probe is removed, the holder has an opening that allows direct visual monitoring of the access site. Alternately, the device may have a removable holder structure to allow greater visual access while maintaining the neck of the patient in the semi or fully mobilized state.

In a method of use, the external carotid compression device 105 is used to achieve hemostasis of a carotid access site. In an alternate embodiment, the external carotid compression device 105 is used to prevent or reduce oozing at a site after a vessel closure device has been used to close the carotid access site. In a further embodiment, the external carotid compression device 105 is used in conjunction with a topical hemostasis patch or pad.

In another embodiment, the external carotid compression device 105 comprises a holder structure for an ultrasound probe wherein the probe provides feedback on access site location and hemostasis. In a further embodiment, the probe additionally provides the compressive force. Alternately, the holder itself provides the compressive force as in the embodiments of FIG. 5 or 6.

In a further method of use, the external carotid compression device restricts the movement of the patient's neck to maintain accurate pressure on the access site and minimize risk of oozing or bleeding at the arterial access site.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A device for applying pressure to a region of a person's neck, comprising:
a belt having a length sized to fit around the neck; and
a compression element coupled to the belt, the compression element sized and shaped to exert a compressive force onto an area of the neck when the belt is tensioned.

2. A device as in claim 1, further comprising a support structure coupled to the belt on a location opposite the location of the compression element.

3. A device as in claim 2, wherein the support structure is an elongated body positioned along the belt.

4. A device as in claim 1, wherein the compression element is an ultrasound probe.

5. A device as in claim 4, wherein the system is configured to emit an alarm, the alarm being based on a signal from the ultrasound probe.

6. A device as in claim 1, further comprising a holder structure that attaches an ultrasound probe to the belt.

7. A device as in claim 6, wherein the position of the ultrasound probe is adjustable within the holder structure.

8. A device as in claim 7, wherein the position of the ultrasound probe is adjustable within the holder structure after the belt is tensioned.

9. A device as in claim 6, wherein the ultrasound probe is the compression element.

10. A device as in claim 6, wherein the holder structure is the compression element.

11. A device as in claim 4, wherein the position of the probe is adjustable based on an ultrasound signal from the probe.

12. A device as in claim 4, wherein tension in the belt is controlled based on an ultrasound signal from the probe.

13. A device as in claim 1, further comprising an inflatable tip on the compression element.

14. A device as in claim 13, wherein the compression element is a holder for an ultrasound probe.

15. A device as in claim 14, wherein an inflation volume of the inflatable tip is controlled based on an ultrasound signal from the probe.

16. A device as in claim 1, wherein the device is configured to restrict movement of the patient's head and neck.

17. A device as in claim 1, wherein the device allows visual monitoring of an access site on the neck when the compressive element is removed.

18. A device as in claim 1, wherein the device allows visual monitoring of an access site on the neck when the compressive element is still compressing the neck.
